(19) United States
(12) Patent Application Publication
(10) Pub. No.: US 2013/0137585 A1
(43) Pub. Date: May 30, 2013

(54) NEW COMBINATION OF EIGHT RISK ALLELES ASSOCIATED WITH AUTISM

(71) Applicants: Jerome Carayol, Issy les Moulineaux (FR); Francis Rousseau, Savigny sur Orge (FR)

(72) Inventors: Jerome Carayol, Issy les Moulineaux (FR); Francis Rousseau, Savigny sur Orge (FR)

(21) Appl. No.: 13/643,311

(22) PCT Filed: May 4, 2011
(86) PCT No.: PCT/EP2011/057148

§ 371 Date: Feb. 14, 2013

Related U.S. Application Data
(60) Provisional application No. 61/331,042, filed on May 4, 2010.

(51) Int. Cl. C12Q 1/68

(52) U.S. Cl. CPC C12Q 1/6883
USPC 506/2; 435/6.11

(57) ABSTRACT

The invention relates to a method of detecting the presence of or predisposition to autism, or to an autism spectrum disorder in a subject, the method comprising detecting the combined presence of an alteration in the gene loci of at least PITX1, ATP2B2, EN2, JARID2, MARK1, ITGB3, CNTNAP2, and HOXA1 in a sample from said subject.

The present invention relates to a method for detecting the presence or predisposition to autism, by detecting a combination of risk alleles in several genes simultaneously.

### BACKGROUND OF THE INVENTION

The Pervasive Developmental disorders (PDDs) referred here as “autism” are a heterogeneous group of disorders characterized by impairments in social interaction, deficits in verbal and nonverbal communication, restricted interests, and repetitive behaviors. The disorders included in the spectrum are Pervasive Developmental disorder, Not Otherwise Specified (PDD-NOS), Autistic disorder, Childhood Disintegrative disorder, Asperger syndrome, and Rett syndrome. Autism spectrum disorder (ASD) represents three of the PDDs: Autistic disorder (AUT), Asperger syndrome (AS), and PDD-NOS.

The ASDs are currently diagnosed through behavioral tests (e.g. Autism Diagnostic Observation Schedule-Generic [ADOS-G]) or indirect, interview-based tests with third parties (e.g., Autism Diagnostic Interview—Revised [ADI-R]) (Lord et al. 1994). However, these tests cannot be applied before a child has reached age 24 months or more. Many children are not diagnosed until much later because the tests are laborious and require specialized training

The prevalence of ASD is estimated at 0.2%, with males being more likely to have a diagnosis than females (male to female ratio of approximately 4:1). Recent studies that have examined the whole spectrum of pervasive developmental disorders have consistently provided estimates in the 60-70/10,000 range, making ASD one of the most frequent childhood neuro developmental disorder (Pediatr Res. 2009 June; 65(6):591-8. Epidemiology of pervasive developmental disorders.Fombonne E.)

ASD has a considerable genetic component, and siblings of autistic children have on average a recurrence risk of approximately 10%. Monozygotic and dizygotic twin studies have shown that autism has a significant genetic component with monozygotic twin concordance rates as high as 91% if broad diagnostic criteria are applied. ASD does not follow a simple Mendelian inheritance pattern and this is thought to be due to the involvement of multiple genes (Veenstra-VanderWeele et al. 2004) with evidence for sex-specific risk alleles in ASD (Stone et al. 2004).

Spontaneous mutations or rare inherited variants may help to explain etiology for a minority of cases, the inheritance pattern of common variants is likely central to disease risk in a majority of multiplex families.

There is no drug therapy available for ASD, although some autistic individuals have been treated with anti-depressant drugs (e.g. Prozac) for secondary symptoms. The main treatments proposed are based on intensive educational programs. Applied early enough some studies show that as many as 50% of autistic children participating in those programs can be referred back to normal schooling and education. The age at which the therapy is proposed is of significant importance. Ideally the programs should start at 18 months age. As outlined above the ADI-R cannot be used for diagnosis under the age of 18 months. Indeed, for infra-structural (availability of trained experts, in the US only 10% of suspected autistic children have direct access to specialists able to carry out ADI-R) and social reasons the average age of diagnosis is 5 years in the US and 8 years in France. A genetic test would have a huge impact, because the test can easily be applied at any age and can be used for pre-screening of individuals for eligibility for an ADI-R, thereby substantially shortening the time from diagnosis to treatment.

### SUMMARY OF THE INVENTION

ASD is highly influenced by genetic factors. Several genes associated with ASD have been identified by academic groups and through in-house research efforts at IntegraGen SA (IntegraGen). However, the contribution to disease risk of each individual gene identified is generally low, and the odds ratio per risk allele rarely is above 1.5. Thus, the predictive power for each gene individually is too small to be of clinical utility in complex diseases. In complex disease states such as type 2 diabetes (Weedon et al. 2006; Lango et al. 2008; Lyssenko et al. 2005; Lu et al. 2005; Lin et al. 2009), cancer (Zheng et al. 2008; Gail 2008), or cardiovascular disease (Kathiresan et al. 2008; Martinelli et al. 2008; Morrison et al. 2007; Humphries et al. 2004), the accumulation of multiple risk alleles markedly increases the risk of being affected, and allows the identification of subgroups of individuals with risk significantly greater than when single nucleotide polymorphisms (SNPs) are studied independently.

The invention described here led to the identification and choice of a combination of specific polymorphisms within eight genes shown previously to be associated with ASD (PITX1, ATP2B2, EN2, JARID2, MARK1, ITGB3, CNTNAP2, and HOXA1).

Using defined variation at these loci, the inventors tested association with clinical diagnosis in a subset of the AGRE cohort comprising about 900 cases stratified according to their gender. Thus, association to ASD was tested in males for ATP2B2, PITX1, HOXA1, CNTNAP2, JARID2 and EN2 and in females for MARK1, ITGB3, CNTNAP2, JARID2 and EN2. Based on these data the inventors have developed a multigene autism risk assessment model specific to the gender. In particular, genotyping these eight genes can allow the estimation of a predictive value for the risk of developing ASD in yet non-diagnosed siblings of affected individuals.

The inventors showed that the predictive value that is obtained by detecting combinations of polymorphisms in these genes is superior to the predictive value obtained when observing alterations in each gene separately, demonstrating its clinical validity.

The clinical utility of this test resides in its ability to select at risk individuals for earlier down-stream diagnosis using psychological profiling tests (e.g. ADI-R or ADOS). The test may also be used in affected individuals to accompany these profiling tests to substantiate the diagnosis for ASD and distinguish it from other psychiatric conditions.

### DETAILED DESCRIPTION OF THE INVENTION

The invention provides a method of detecting the presence of or predisposition to autism, preferably to an autism spectrum disorder or to an autistic disorder, in a subject, the method comprising detecting the presence of an alteration in the gene loci of at least PITX1, ATP2B2, EN2, JARID2, MARK1, ITGB3, CNTNAP2, and HOXA1 in a sample from said subject. In a preferred embodiment, the alteration is a single nucleotide polymorphism.

Unless otherwise specified, the term “autism” refers to Autism spectrum disorder (ASD) which is a heterogeneous group of disorders characterized by impairments in social interaction, deficits in verbal and nonverbal communication, restricted interests, and repetitive behaviors. Autism spectrum disorder (ASD) are preferably targeted, including Autistic disorder (AUT), Asperger syndrome (AS), and other pervasive developmental disorders Not Otherwise Specified (PPD-NOS). ASD is construed as any condition of impaired social interaction and communication with restricted repetitive and stereotyped patterns of behavior, interests and activities present before the age of 3, to the extent that health may be impaired. The invention provides diagnostic screening methods based on a monitoring of several genes in a subject. The subject may be at early, pre-symptomatic stage, or late stage. The subject may be any human male or female, preferably a child or a young adult. The subject can be asymptomatic.

The method is particularly useful when the subject is a sibling of an individual with autism or an autism-spectrum disorder, i.e. an individual already diagnosed with autism or an autism spectrum disorder. The likelihood that a sibling of a child with autism also develops autism or an autism-associated disorder is between 5 and 10 percent (Szatmari et al., 2007). This is approximately 20 times greater than the rate at which autism affects individuals who are not related to an affected individual. The method of the invention can be performed at any age after birth and used to pre-screen individuals requiring further assessment with the ADI-R, shortening the time from diagnosis to intervention.

The diagnosis methods can be performed in vitro, ex vivo or in vivo, preferably in vitro or ex vivo. They use a sample from the subject. The sample may be any biological sample derived from a subject, which contains nucleic acids. Examples of such samples include fluids, tissues, cell samples, organs, biopsies, etc. Most preferred samples are blood, plasma, saliva, jugal cells, urine, seminal fluid, etc. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids may be pre-purified or enriched by conventional techniques, and/or reduced in complexity. Nucleic acids may also be treated with enzymes or other chemical or physical treatments to produce fragments thereof. Considering the high sensitivity of the claimed methods, very few amounts of sample are sufficient to perform the assay.

The sample is preferably contacted with reagents such as probes, or primers in order to assess the presence of an altered gene locus. Contacting may be performed in any suitable device, such as a plate, tube, well, glass, etc. In specific embodiments, the contacting is performed on a substrate coated with the reagent, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample. The finding of a specific allele of PITX1, ATP2B2, EN2, JARID2, MARK1, ITGB3, CNTNAP2, and HOXA1 DNA in the sample is indicative of the presence of a gene locus variant in the subject, which can be correlated to the presence, predisposition or stage of progression of autism, or an autism spectrum disorder. For example, an individual having a germ line mutation has an increased risk of developing autism, an autism spectrum disorder, or an autism-associated disorder. The determination of the presence of an altered gene locus in a subject also allows the design of appropriate therapeutic intervention, which is more effective and customized. Also, this determination at the pre-symptomatic level allows a preventive regimen to be applied.

An alteration in a gene locus may be any form of mutation(s), deletion(s), rearrangement(s) and/or insertions in the coding and/or non-coding region of the locus, alone or in various combination(s). Alterations more specifically include point mutations or single nucleotide polymorphisms (SNP). Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The gene locus alteration may result in the creation of stop codons, frameshift mutations, amino acid substitutions, particular RNA splicing or processing, product instability, truncated polypeptide production, etc. The alteration may result in the production of a polypeptide with altered function, stability, targeting or structure. The alteration may also cause a reduction in protein expression or, alternatively, an increase in said production.

Once a first SNP has been identified in a genomic region of interest, the practitioner of ordinary skill in the art can easily identify additional SNPs in linkage disequilibrium with this first SNP. Indeed, any SNP in linkage disequilibrium with a first SNP associated with autism or an associated disorder will be associated with this trait. Therefore, once the association has been demonstrated between a given SNP and autism or an associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnostic methods according to the present invention.

PITX1, ATP2B2, EN2, JARID2, MARK1, ITGB3, CNTNAP2, and HOXA1 Genes

International patent application WO2006/003520 discloses that the PITX1 gene on chromosome 5 and certain alleles thereof are related to susceptibility to autism. As used herein, the term “PITX1 gene” designates the pituitary homeobox transcription factor 1 gene on human chromosome 5q31.1, as well as variants, analogs and fragments thereof, including alleles thereof (e.g., germline mutations) which are related to susceptibility to autism and autism-associated disorders. The PITX1 gene may also be referred to as paired-like homeodomain transcription factor pituitary homeobox 1, or PTX1.

International patent application WO2006/100608 describes that the ATP2B2 gene on chromosome 3 and certain alleles thereof are related to susceptibility to autism. As used herein, the term “ATP2B2 gene” designates the ATPase, Ca++ transporting, plasma membrane 2 gene on human chromosome 3p25.3, as well as variants, analogs and fragments thereof, including alleles thereof (e.g., germline mutations) which are related to susceptibility to autism and autism-associated disorders. The ATP2B2 gene may also be referred to as PMCA2. Association of ATP2B2 gene with autism was also reported in Hu et al. 2009.

International patent application WO2005/007812 discloses that the EN2 gene on chromosome 7q36.3 and certain alleles thereof are related to susceptibility to autism. This gene is name after “ENGRAILED 2”, a homeobox transcription factor. Association of EN2 with autism was also reported in Cheh et al. 2006 and Wang et al. 2008.

In previous studies, rs6872664 (PITX1), rs35678 (ATP2B2), rs2292813 (SLC25A12), and rs1861972 (EN2) showed significant association with autism with relative risks varying with the gene, the definition of autism, and the genotype (heterozygous or homozygous) (Philippi et al, 2007; WO2006/100608, Ramoz et al, 2004; Benayed et al, 2005).

In a genome wide association study on autism, Weiss et al, 2009, identified a single nucleotide polymorphism in JARID2 (rs7766973), a gene already associated to schizophrenia (Pedrosa et al., 2007; Liu et al., 2009), another psychiatric disease that shares a common genetic background with autism (Crespi et al., 2009; Carrol et al., 2009). JARID2, a member of the ARID (AT-rich interaction domain) family of transcription modulators, is an ortholog of the mouse jumonji gene, which encodes a nuclear protein essential for mouse embryogenesis, including neural tube formation. Overexpression of mouse jumonji negatively regulates cell proliferation. The jumonji proteins contain a DNA-binding domain, called an AT-rich interaction domain (ARID), and share regions of similarity with human retinoblastoma-binding protein-2 and the human SMCX protein.

International patent application WO2006/087634 describes that the MARK1 gene on chromosome 1 and certain alleles thereof are related to susceptibility to autism. As used herein, the term “MARK1 gene” designates the MAP/microtubule affinity-regulating kinase 1 gene on human chromosome 1q41, as well as variants, analogs and fragments thereof, including alleles thereof (e.g., germline mutations) which are related to susceptibility to autism and autism-associated disorders. The MARK1 gene may also be referred to as MAP/microtubule affinity-regulating kinase, MARK, and KIAA1477. The association of MARK1 with autism was also reported in Maussion et al. 2008, using a family based association study and an expression analysis.

The ITGB3 gene encodes ITGB3 protein product is the integrin beta chain beta 3. Integrin beta 3 is found along with the alpha IIb chain in platelets. Integrins are known to participate in cell adhesion as well as cell-surface mediated signalling. Association of ITGB3 with autism is reported in Weiss et al. 2006; Coutinho et al. 2007; Ma et al. 2009.

International patent application WO2006/0568739 describes that the CNTNAP2 gene on chromosome 7 and certain alleles thereof are related to susceptibility to autism. As used herein, the term “CNTNAP2 gene” designates the contactin associated protein-like 2 gene on chromosome 7q35-q36, as well as variants, analogs and fragments thereof, including alleles thereof (e.g., germline mutations) which are related to susceptibility to obesity and associated disorders. The CNTNAP2 gene may also be referred to as contactin-associated protein 2, cell recognition molecule (CASPR2), homolog of Drosophilia neurexin IV (NRXN4). Association of CNTNAP2 with autism was also reported in Alarcon et al. 2008; Arking et al. 2008; Poot et al. 2009.

U.S. Pat. No. 6,228,582 describes that polymorphisms in HOXA1 gene are useful genetic markers for autism. In vertebrates, the genes encoding the class of transcription factors called homeobox genes (HOX) are found in clusters named A, B, C, and D on four separate chromosomes. Expression of these proteins is spatially and temporally regulated during embryonic development. HOXA1 is part of the A cluster on chromosome 7 and encodes a DNA-binding transcription factor which may regulate gene expression, morphogenesis, and differentiation. The encoded protein may be involved in the placement of hindbrain segments in the proper location along the anterior-posterior axis during development. Association of HOXA1 with autism was mentioned in Ingram et al. 2000; Conciatori et al. 2004; Sen et al. 2007.

More specifically, the inventors showed that a specific combination of eight single nucleotide polymorphisms (SNPs) allowed to obtain a predictive power that is clinically very useful for detecting autism or a autism-spectrum disorder. These SNPs are shown in Table 1.

TABLE 1
Autism-associated SNPs in combination

| | | Autism- | Deleterious | |
|---|---|---|---|---|
| | | associated | allele | |
| | | risk | frequency | |
| Gene | SNP name | allele | (HapMap) | SEQ ID NO: |
| PITX1 | rs6872664 | 1 = C | 0.93 | 1 (nucleotide 301) |
| ATP2B2 | rs2278556 | 1 = A | 0.38 | 2 (nucleotide 201) |
| EN2 | rs1861972 | 1 = A | | 3 (nucleotide 301) |
| JARID2 | rs7766973 | 1 = C | 0.63 | 4 (nucleotide 251) |
| MARK1 | rs12410279 | 1 = A | 0.87 | 5 (nucleotide 201) |
| ITGB3 | rs5918 | 2 = T | 0.86 | 6 (nucleotide 401) |
| CNTNAP2 | rs7794745 | 2 = T | 0.31 | 7 (nucleotide 301) |
| HOXA1 | rs10951154 | 2 = T | 0.82 | 8 (nucleotide 521) |

A subject of the invention is thus a method of detecting the presence of or predisposition to autism, or to an autism spectrum disorder in a subject, the method comprising detecting the combined presence of an alteration in the gene loci of at least PITX1, ATP2B2, EN2, JARID2, MARK1, ITGB3, CNTNAP2, and HOXA1 in a sample from said subject.

In a embodiment the method comprises detecting the presence of a single nucleotide polymorphism (SNP) at position rs6872664 of PITX1 (nucleotide 301 on SEQ ID NO:1), and/or detecting the presence of a single nucleotide polymorphism (SNP) at position rs2278556 of ATP2B2 (nucleotide 201 on SEQ ID NO:2), and/or detecting the presence of a single nucleotide polymorphism (SNP) at position rs1861972 of EN2 (nucleotide 301 on SEQ ID NO:3), and/or detecting the presence of a single nucleotide polymorphism (SNP) at position rs7766973 of JARID2 (nucleotide 251 on SEQ ID NO:4) and/ordetecting the presence of a single nucleotide polymorphism (SNP) at position rs12410279 of MARK1 (nucleotide 201 on SEQ ID NO:5) and/or detecting the presence of a single nucleotide polymorphism (SNP) at position rs5918 of ITGB3 (nucleotide 401 on SEQ ID NO:6) and/or detecting the presence of a single nucleotide polymorphism (SNP) at position rs7794745 of CNTNAP2 (nucleotide 301 on SEQ ID NO:7) and/or detecting the presence of a single nucleotide polymorphism (SNP) at position rs10951154 of HOXA1 (nucleotide 521 on SEQ ID NO:8).

In a particularly preferred embodiment, the method comprises detecting the simultaneous presence of a SNP at position rs6872664 of PITX1 (nucleotide 301 on SEQ ID NO:1), position rs2278556 of ATP2B2 (nucleotide 201 on SEQ ID NO:2), position rs1861972 of EN2 (nucleotide 301 on SEQ ID NO:3), position rs7766973 of JARID2 (nucleotide 251 on SEQ ID NO:4), position rs12410279 of MARK1 (nucleotide 201 on SEQ ID NO:5), position rs5918 of ITGB3 (nucleotide 401 on SEQ ID NO:6), position rs7794745 of CNTNAP2 (nucleotide 301 on SEQ ID NO:7), and position rs10951154 of HOXA1 (nucleotide 521 on SEQ ID NO:8),

wherein detection of the simultaneous presence of C at position rs6872664 of PITX1 (nucleotide 301 on SEQ ID NO:1), A at position rs2278556 of ATP2B2 (nucleotide 201 on SEQ ID NO:2), A at position rs1861972 of EN2 (nucleotide 301 on SEQ ID NO:3), C at position rs7766973 of JARID2 (nucleotide 251 on SEQ ID NO:4), A at position rs12410279 of MARK1 (nucleotide 201 on SEQ ID NO:5), T at position rs5918 of ITGB3 (nucleotide 401 on SEQ ID NO:6), T at position rs7794745 of CNTNAP2 (nucleotide 301 on SEQ ID NO:7), and T at position rs10951154 of HOXA1 (nucleotide 521 on SEQ ID NO:8), is indicative of the presence of or predisposition to autism.

In another embodiment, the presence of SNPs in linkage disequilibrium (LD) with the above-identified SNPs may be detected, in place of, or in addition to, said identified SNPs (Table 2).

TABLE 2
Identification of SNPs in LD using HapMap data information and
tagging coefficient r² = 1.00 (complete linkage disequilibrium):

| | | Orientation | deleterious allele oriented | |
|---|---|---|---|---|
| | dbSNP ID (submitted SNP, ss | strand in | in strand + (frequency | SEQ ID NO: (all sequences |
| Gene | ID oriented in strand+) | dbSNP b126 | HapMap-Ceuc Population) | are oriented on strand+) |
| PITX1 | rs1700488 (ss44653899) | + | G (0.90) | 9 (nucleotide 301) |
| | rs6596189 (ss10226076) | + | C (0.86) | 10 (nucleotide 201) |
| | rs11959298 (ss44589780) | + | A (0.91) | 11 (nucleotide 301) |
| | rs6596188 (ss10225732) | + | A (0.92) | 12 (nucleotide 301) |
| | rs1131611 (ss13907917) | − | G (0.86) | 13 (nucleotide 201) |
| | rs6871427 (ss10214017) | + | G (0.90) | 14 (nucleotide 201) |
| | rs10079987 (ss13933598) | + | T (0.86) | 15 (nucleotide 201) |
| | rs254549 (ss330962) | − | A (0.86) | 16 (nucleotide 101) |
| ATP2B2 | rs17223473 (ss524208) | + | T (0.38) | 17 (nucleotide 452) |
| MARK1 | rs3806329 (ss44063993) | − | A (0.87) | 23 (nucleotide 301) |
| ITGB3 | rs7214096 (ss10858974) | + | G (0.86) | 24 (nucleotide 343) |
| | rs8069732 (ss12393719) | + | C (0.86) | 25 (nucleotide 251) |

The method of the invention, also referred to as “the test” thus preferably includes genotyping of all eight genes. The test can be used to strengthen the diagnosis by confirming a known risk profile. In such case a negative test result does not invalidate the diagnosis for autism.

Alternatively the test can be used to establish a detailed risk profile for the non-diagnosed sibling. Possible outcomes are:
Presence of a risk allele in one or more genes, heterozygous or homozygous implicating increased risk
Absence of a risk allele in the un-diagnosed sibling and/or the autistic sibling. In this case no risk profile can be established.

The presence of an alteration in the gene locus may be detected by sequencing, selective hybridisation and/or selective amplification.

Sequencing can be carried out using techniques well known in the art, using automatic sequencers. The sequencing may be performed on the complete genes or, more preferably, on specific domains thereof, typically those known or suspected to carry deleterious mutations or other alterations.

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the gene or locus are able to specifically hybridize with a portion of the gene locus that flank a target region of said locus, said target region being altered in certain subjects having autism, an autism spectrum disorder, or an autism-associated disorder

Hybridization detection methods are based on the formation of specific hybrids between complementary nucleic acid sequences that serve to detect nucleic acid sequence alteration(s). A particular detection technique involves the use of a nucleic acid probe specific for wild type or altered gene, followed by the detection of the presence of a hybrid. The probe may be in suspension or immobilized on a substrate or support (as in nucleic acid array or chips technologies). The probe is typically labelled to facilitate detection of hybrids.

In a most preferred embodiment, an alteration in the gene locus is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the alteration of the genes, a sample from a test subject is labelled and contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art (see e.g. the review by Kidgell&Winzeler, 2005 or the review by Hoheisel, 2006).

The example illustrates the present invention without limiting its scope.

### EXAMPLE 1

### Autism Risk Prediction in Children

Materials and methods

Population:

The population consists in 482 informative families from a subset of AGRE repository with at least one affected (ASD) children genotyped: 87 are trios including the parents and only the index case, 351 are families with two affected siblings, 40 are families with 3 affected siblings and 4 are families with 4 affected siblings. In these families, there is a total of 838 cases with ASD genotyped together with their parents for all eight genes investigated. The male:female sex ratio is 3.45:1 in this sample with 717 males and 208 females affected

Methods

Genotyping

Samples were genotyped using TaqMan allele discrimination assays supplied by Applied Biosystems (Foster City, Calif., USA). Genotyping was performed on 384 well plates in a final volume of 5 μl with 2 μl of genomic DNA at 5 ng/μl, 0.125 μl of 40× SNP TaqMan Assay mix, 2.5 μl of TaqMan Genotyping Master Mix and 0.375 μl of dH₂O in each well. PCR was then carried out using a 9700 Gene Amp PCR System (Applied Biosystems) with a profile of 95° C. for 10 min and then 40 cycles at 92° C. for 15 sec and 60° C. for 60 sec. Plates were then subjected to end-point read in a 7900 Real-Time PCR System (Applied Biosystems). The results were first evaluated by cluster variations; the allele calls were then assigned automatically. Genotyping and data analysis were blinded to patient identification. Signal intensity plots and missing genotype frequencies were used for investigating genotyping quality. Poor clustering and missing fractions 5% per SNP lead to regenotyping. Genotyping success rate was 97.4%. Parents were genotyped to check for Mendelian inconsistencies and to verify family relationships.

Statistical Method and Results:

Association was tested using an additive model for all the genes, with the genotype homozygous non carrier of the risk allele coded 0, the heterozygous genotype coded 1, and the genotype homozygous carrier coded 2 except for ATP2B2 for which, according to published data, a recessive model was tested with the homozygous carrier genotype coded 2 and the two other genotypes coded 0.

All these analyses were done using the Pedigree Disequilibrium Test (PDT) implemented in the UNPHASED software that deals with missing data, test for gender effect and gene—gene interaction excepted for ATP2B2. ATP2B2 is associated to ASD under a recessive assumption (Philippi et al. 2007) and UNPHASED doesn't allow analysis of model other than the additive model. Association of this gene was conducted using an approach proposed by Cordell et al. (2002, 2004) that did not deal with missing data but allow the analysis under the recessive model assumption. Because this gene has already been associated to autism in previous studies, over-transmission of the risk allele only was tested with a one-sided test. CNTNAP2, JARID2 and EN2 were tested in the whole sample (i.e. without gender stratification) since they entered in both gender specific tests. ATP2B2, PITX1 and HOXA1 were tested in males only and MARK1 and ITGB3 in females only. Replication of the association in the specific sample was declared at the nominal level (p=0.05). Results are presented in Table 3. The inventors observed that all SNPs were associated at the nominal level in their specific sample.

The risk score (RS) for an individual is defined as the sum of deleterious alleles for the gender specific genes observed for this individual. Thus, in males, 0 (no risk allele) to 12 risk alleles (all risk alleles for the 6 genes specific to males) may be observed which corresponds to a risk score (RSₘₐₗₑ) that may varied between 0 and 12. And, in females, 0 (no risk allele) to 10 risk alleles (all risk alleles for the 5 genes specific to females) may be observed which corresponds to a risk score (RS_{female}) that may varied between 0 and 10. The data were analyzed using the case-pseudocontrol approach proposed by Cordell (Cordell, 2004; Cordell et al., 2004) since no unaffected sibling were available in the present AGRE sample. For each child with ASD, a pseudocontrol was constructed from parental untransmitted alleles to the child with autism. Then, the data are analyzed as in a classical matched case-control study using a conditional logistic regression to estimate genetic relative risk (that is equivalent to odds ratios (ORs) in diseases with low prevalence as ASD. The term OR was used in the next sections instead of genetic relative risk), 95% confidence intervals and associated p values. For each RS value (i.e. threshold), sensitivity (defined as the probability in ASD case to have a RS greater or equal to a specific value) and specificity (defined as the probability in “pseudocontrols” to have a RS strictly smaller than a specific value) are estimated as the odds ratio (OR) that correspond to the OR of individuals with a RS value greater than the threshold compared to individuals with a RS strictly smaller than this threshold value. Analyses for RSₘₐₗe were conducted in the sample including only ASD males and RS_{female} in the sample including only ASD females. Results are provided in Tables 4 and 5.

Results

TABLE 3
Association results using the PDT implemented in UNPHASED
software (excepted for ATP2B2). One sided test p values
are provided assuming replication tests of an over-transmission
of a deleterious allele to cases.

| Gene | SNP ID | p value | Sample |
|---|---|---|---|
| PITX1 | rs6872664 | 0.003 | Males only |
| ATP2B2 | rs2278556 | 0.0162 | Males only |
| HOXA1 | rs10951154 | 0.045 | Males only |
| EN2 | rs1861972 | 0.0075 | Whole sample |
| CNTNAP2 | rs7794745 | 0.000025 | Whole sample |
| JARID2 | rs7766973 | 0.004 | Whole sample |
| MARK1 | rs12410279 | 0.009 | Females only |
| ITGB3 | rs5918 | 0.015 | Females only |

TABLE 4
Sensitivity/specificity, with their 95% confidence intervals
(CI), odds ratio (OR), and its corresponding p value associated
to each RSₘₐₗₑ value for ASD in males

| Number of | | | | |
|---|---|---|---|---|
| Risk alleles | Sensitivity | Specificity | | |
| (RSₘₐₗₑ) | (95% CI) | (95% CI) | OR | p. value |
| 3 | 1.00 | 0.00 | — | — |
| | — | — | | |
| 4 | 1.00 | 0.01 | 4.5 | 0.02 |
| | 0.99-1.00 | 0.00-0.043 | | |
| 5 | 0.97 | 0.04 | 1.8 | 0.23 |
| | 0.96-0.98 | 0.02-0.06 | | |
| 6 | 0.90 | 0.19 | 2.0 | 0.0001 |
| | 0.85-0.94 | 0.15-0.23 | | |
| 7 | 0.75 | 0.42 | 2.2 | 0.000001 |
| | 0.71-0.79 | 0.36-0.47 | | |
| 8 | 0.47 | 0.65 | 1.6 | 0.0001 |
| | 0.42-0.52 | 0.60-0.70 | | |
| 9 | 0.23 | 0.86 | 1.8 | 0.0005 |
| | 0.19-0.27 | 0.83-0.89 | | |
| 10 | 0.08 | 0.95 | 1.7 | 0.028 |
| | 0.05-0.11 | 0.93-.97 | | |
| 11 | 0.02 | 0.98 | 0.9 | 0.69 |
| | 0.01-0.03 | 0.97-0.99 | | |
| 12 | 0.00 | 1.00 | — | — |
| | — | — | | |

TABLE 5
Sensitivity/specificity, with their 95% confidence intervals
(CI), odds ratio (OR), and its corresponding p value associated
to each RS_{female} value for ASD in females

| Number of | | | | |
|---|---|---|---|---|
| Risk alleles | Sensitivity | Specificity | | |
| (RS_{female}) | (95% CI) | (95% CI) | OR | p. value |
| 3 | 1.00 | 0.00 | — | — |
| | — | — | | |
| 4 | 1.00 | 0.00 | — | — |
| | — | — | | |
| 5 | 0.96 | 0.06 | 1.5 | 0.46 |
| | 0.92-1.00 | 0.03-0.010 | | |
| 6 | 0.89 | 0.20 | 2.0 | 0.03 |
| | 0.84-0.94 | 0.15-0.25 | | |
| 7 | 0.71 | 0.48 | 2.3 | 0.0004 |
| | 0.64-0.78 | 0.40-0.56 | | |
| 8 | 0.41 | 0.80 | 2.7 | 0.00006 |
| | 0.33-0.48 | 0.74-0.86 | | |
| 9 | 0.18 | 0.94 | 3.1 | 0.004 |
| | 0.12-0.23 | 0.89-0.98 | | |
| 10 | 0.03 | 0.99 | 2.5 | 0.27 |
| | 0.00-0.05 | 0.97-1.00 | | |

In Table 1 and 2, instead of 3 (2 for ATP2B2) possible states with a limited choice of sensibility and specificity to define a test (sensitivity and specificity values distribution for each SNP are provided in Table 6 in males and Table 7 in females), RSs allowed a large choice of RS threshold to define a test in males and in females separately according to appropriate sensitivity and specificity values. In complex disease such as autism, it is important that “risk assessment test” maintained a high specificity (greater than 80%).

In Table 6 and 7, we can see that SNPs are associated to low specificity generally smaller than 80% excepted for ATP2B2 in males and CNTNAP2. But, for these two exceptions, the sensitivity remained low (smaller than 20%). In female, the RS takes values from 3 to 10 with different ratios of sensitivity/specificity. A threshold of 8 risk alleles allows to build a test with 41% sensitivity and 80% specificity with an elevated OR=2.73 (p value 0.00006) largely greater than OR values observed in single SNP (generally smaller than 1.5 and rarely exceeding 2.00). Such sensitivity / specificity ratio was never reached with single SNPs (Table 7) where the specificity remained low excepted for CNTNAP2 (86%) but with a low corresponding sensitivity of 15%. In males, the same effect was observed in a lesser extend. In males, RSₘₐₗₑ ranges from 3 to 12 with an interesting sensitivity/specificity ratio of 23%/86% for a RS threshold of 9 associated to a moderate but highly significant OR=1.8 (p value=0.0005). When maintaining a high specificity (i.e. greater than 80%), none of the single SNPs reached such interesting sensitivity/specificity ratio (Table 7) except for ATP2B2 SNP with a specificity of 86% but with a low sensitivity under 20% (18%).

TABLE 6
Sensitivity and specificity values with 95% confidence
interval for SNPs in the RS for males

| Gene | RS | sensitivity | specificity |
|---|---|---|---|
| JARID2 | 0 | 1.00 | 0.00 |
| | 1 | 0.99 [0.98-1.00] | 0.19 [0.16-0.22] |
| | 2 | 0.81 [0.75-0.86] | 0.68 [0.63-0.73] |
| CNTNAP2 | 0 | 1.00 | 0.00 |
| | 1 | 0.85 [0.81-0.89] | 0.19 [0.16-0.22] |
| | 2 | 0.38 [0.33-0.42] | 0.68 [0.63-0.73] |
| EN2 | 0 | 1.00 | 0.00 |
| | 1 | 0.94 [0.92-0.96] | 0.09 [0.07-0.12] |
| | 2 | 0.52 [0.47-0.57] | 0.49 [0.45-0.54] |
| ATP2B2 | 0 | 1.00 | 0.00 |
| | 2 | 0.18 [0.14-0.23] | 0.86 [0.82-0.90] |
| PITX1 | 0 | 1.00 | 0.00 |
| | 1 | 0.99 [0.96-1.00] | 0.03 [0.01-0.04] |
| | 2 | 0.81 [0.78-0.85] | 0.24 [0.19-0.28] |
| HOXA1 | 0 | 1.00 | 0.00 |
| | 1 | 0.98 [0.95-1.00] | 0.03 [0.01-0.04] |
| | 2 | 0.75 [0.71-0.80] | 0.29 [0.25-0.34] |

TABLE 7
Sensitivity and specificity values with 95% confidence
interval for SNPs in the RS for females

| Gene | RS | sensitivity | specificity |
|---|---|---|---|
| JARID2 | 0 | 1.00 | 0.00 |
| | 1 | 0.85 [0.79-0.90] | 0.20 [0.14-0.26] |
| | 2 | 0.39 [0.32-0.47] | 0.66 [0.59-0.73] |
| CNTNAP2 | 0 | 1.00 | 0.00 |
| | 1 | 0.64 [0.57-0.72] | 0.45 [0.37-0.54] |
| | 2 | 0.15 [0.09-0.20] | 0.87 [0.81-0.93] |
| EN2 | 0 | 1.00 | 0.00 |
| | 1 | 0.93 [0.89-0.98] | 0.09 [0.05-0.14] |
| | 2 | 0.60 [0.52-0.67] | 0.54 [0.46-0.63] |
| MARK1 | 0 | 1.00 | 0.00 |
| | 1 | 0.99 [0.97-1.00] | 0.01 [0.00-0.02] |
| | 2 | 0.82 [0.75-0.88] | 0.33 [0.25-0.41] |
| ITGB3 | 0 | 1.00 | 0.00 |
| | 1 | 0.99 [0.98-1.00] | 0.03 [0.01-0.05] |
| | 2 | 0.81 [0.75-0.86] | 0.34 [0.26-0.41] |

### REFERENCES

Alarcon M, Abrahams B S, Stone J L, Duvall J A, Perederiy J V, Bomar J M, Sebat J, Wigler M, Martin C L, Ledbetter D H, Nelson S F, Cantor R M, Geschwind D H (2008) Linkage, association, and gene-expression analyses identify CNTNAP2 as an autism-susceptibility gene. Am J Hum Genet 82:150-159

Arking D E, Cutler D J, Brune C W, Teslovich T M, West K, Ikeda M, Rea A, Guy M, Lin S, Cook E H, Chakravarti A (2008) A common genetic variant in the neurexin superfamily member CNTNAP2 increases familial risk of autism. Am J Hum Genet 82:160-164

Benayed R, Gharani N, Rossman I, et al. Support for the Homeobox Transcription Factor Gene ENGRAILED 2 as an Autism Spectrum Disorder Susceptibility Locus. Am J Hum Genet 2005; 77:851-68.

Carroll L S., Owen M J (2009). Genetic overlap between autism, schizophrenia and bipolar disorder. Genome Med. 1(10):102.

Cheh M A, Millonig J H, Roselli L M, Ming X, Jacobsen E, Kamdar S, Wagner G C (2006) En2 knockout mice display neurobehavioral and neurochemical alterations relevant to autism spectrum disorder. Brain Res 1116:166-176

Conciatori M, Stodgell C J, Hyman S L, O'Bara M, Militerni R, Bravaccio C, Trillo S, Montecchi F, Schneider C, Melmed R, Elia M, Crawford L, Spence S J, Muscarella L, Guarnieri V, D'Agruma L, Quattrone A, Zelante L, Rabinowitz D, Pascucci T, Puglisi-Allegra S, Reichelt K L, Rodier P M, Persico A M (2004) Association between the HOXA1 A218G polymorphism and increased head circumference in patients with autism. Biol Psychiatry 55:413-419

Cordell, H. J., B. J. Barratt, et al. (2004). “Case/pseudocontrol analysis in genetic association studies: A unified framework for detection of genotype and haplotype associations, gene-gene and gene-environment interactions, and parent-of-origin effects.” Genet Epidemiol 26(3): 167-85.

Cordell, H. J. and D. G. Clayton (2002). “A unified stepwise regression procedure for evaluating the relative effects of polymorphisms within a gene using case/control or family data: application to HLA in type 1 diabetes.” Am J Hum Genet 70(1): 124-41.

Coutinho A M, Sousa I, Martins M, Correia C, Morgadinho T, Bento C, Marques C, Ataide A, Miguel T S, Moore J H, Oliveira G, Vicente A M (2007) Evidence for epistasis between SLC6A4 and ITGB3 in autism etiology and in the determination of platelet serotonin levels. Hum Genet 121:243-256

Crespi, B., Stead, P. & Elliot, M. Evolution in health and medicine Sackler colloquium: Comparative genomics of autism and schizophrenia. Proc Natl Acad Sci U S A 107 Suppl 1, 1736-41 (2009).

Gail M H: Discriminatory accuracy from single-nucleotide polymorphisms in models to predict breast cancer risk. J Natl Cancer Inst 2008, 100:1037-1041.Hoheisel, Nature Reviews, Genetics, 2006, 7:200-210

Hu V W, Sarachana T, Kim K S, Nguyen A, Kulkarni S, Steinberg M E, Luu T, Lai Y, Lee N H (2009) Gene expression profiling differentiates autism case-controls and phenotypic variants of autism spectrum disorders: evidence for circadian rhythm dysfunction in severe autism. Autism Res 2:78-97

Humphries S E, Ridker P M, Talmud P J: Genetic testing for cardiovascular disease susceptibility: a useful clinical management tool or possible misinformation? Arterioscler Thromb Vasc Biol 2004, 24:628-636.

Ingram J L, Stodgell C J, Hyman S L, Figlewicz D A, Weitkamp L R, Rodier P M (2000) Discovery of allelic variants of HOXA1 and HOXB1: genetic susceptibility to autism spectrum disorders. Teratology 62:393-405

Kathiresan S, Melander O, Anevski D, Guiducci C, Burtt N P, Roos C, Hirschhorn J N, Berglund G, Hedblad B, Groop L et al.: Polymorphisms associated with cholesterol and risk of cardiovascular events. N Engl J Med 2008, 358:1240-1249.

Kidgell&Winzeler, Chromosome Research, 2005:13:225-235

Lango H, Palmer C N, Morris A D, Zeggini E, Hattersley A T, McCarthy M I, Frayling T M, Weedon M N: Assessing the combined impact of 18 common genetic variants of modest effect sizes on type 2 diabetes risk. Diabetes 2008, 57:3129-3135.

Lin X, Song K, Lim N, Yuan X, Johnson T, Abderrahmani A, Vollenweider P, Stirnadel H, Sundseth S S, Lai E et al.: Risk prediction of prevalent diabetes in a Swiss population using a weighted genetic score—the CoLaus Study. Diabetologia 2009, 52:600-608.

Liu, Y. et al. Whole genome association study in a homogenous population in Shandong peninsula of China reveals JARID2 as a susceptibility gene for schizophrenia. J Biomed Biotechno12009, 536918 (2009).

Lord C, Rutter M, Le Couteur A (1994) Autism Diagnostic Interview-Revised: a revised version of a diagnostic interview for caregivers of individuals with possible pervasive developmental disorders. J Autism Dev Disord 24:659-685

Lu Q, Elston R C: Using the optimal receiver operating characteristic curve to design a predictive genetic test, exemplified with type 2 diabetes. Am J Hum Genet 2008, 82:641-651.

Lyssenko V, Almgren P, Anevski D, Orho-Melander M, Sjogren M, Saloranta C, Tuomi T, Groop L: Genetic prediction of future type 2 diabetes. PLoS Med 2005, 2:e345.

Ma D Q, Rabionet R, Konidari I, Jaworski J, Cukier H N, Wright H H, Abramson R K, Gilbert J R, Cuccaro M L, Pericak-Vance M A, Martin E R (2009) Association and gene-gene interaction of SLC6A4 and ITGB3 in autism. Am J Med Genet B Neuropsychiatr Genet

Martinelli N, Trabetti E, Pinotti M, Olivieri O, Sandri M, Friso S, Pizzolo F, Bozzini C, Caruso P P, Cavallari U et al.: Combined effect of hemostatic gene polymorphisms and the risk of myocardial infarction in patients with advanced coronary atherosclerosis. PLoS ONE 2008, 3:e1523

Maussion G, Carayol J, Lepagnol-Bestel A M, Tores F, Loe-Mie Y, Milbreta U, Rousseau F, Fontaine K, Renaud J, Moalic J M, Philippi A, Chedotal A, Gorwood P, Ramoz N, Hager J, Simonneau M (2008) Convergent evidence identifying MAP/microtubule affinity-regulating kinase 1 (MARK1) as a susceptibility gene for autism. Hum Mol Genet 17:2541-2551

Morrison A C, Bare L A, Chambless L E, Ellis S G, Malloy M, Kane J P, Pankow J S, Devlin J J, Willerson J T, Boerwinkle E: Prediction of coronary heart disease risk using a genetic risk score: the Atherosclerosis Risk in Communities Study. Am J Epidemiol 2007, 166:28-35.

Philippi A, Tores F, Carayol J, Rousseau F, Letexier M, Roschmann E, Lindenbaum P, Benajjou A, Fontaine K, Vazart C, Gesnouin P, Brooks P, Hager J (2007) Association of autism with polymorphisms in the paired-like homeodomain transcription factor 1 (PITX1) on chromosome 5q31: a candidate gene analysis. BMC Med Genet 8:74

Pedrosa, E. et al. Positive association of schizophrenia to JARID2 gene. Am J Med Genet B Neuropsychiatr Genet 144B, 45-51 (2007).

Poot M, Beyer V, Schwaab I, Damatova N, Van't Slot R, Prothero J, Holder S E, Haaf T (2009) Disruption of CNTNAP2 and additional structural genome changes in a boy with speech delay and autism spectrum disorder. Neurogenetics

Ramoz N, Reichert J G, Smith C J, et al. Linkage and association of the mitochondrial aspartate/glutamate carrier SLC25Al2 gene with autism. Am J Psychiatry 2004; 161:662-9.

Sen B, Sinha S, Ahmed S, Ghosh S, Gangopadhyay P K, Usha R (2007) Lack of association of HOXA1 and HOXB1 variants with autism in the Indian population. Psychiatr Genet 17:1

Szatmari P, Paterson A D and the Autism Genome Project Consortium (2007). Mapping autism risk loci using genetic linkage and chromosomal rearrangements. Nat Genet. 39:319-28.

Stone J L, Merriman B, Cantor R M, Yonan A L, Gilliam C, Geshwind D H, Nelson S F (2004). Evidence for sex-specific risk alleles in autism spectrum disorder. Am J Hum Genet. 75:1117-1123.

Veenstra-VanderWeele J, Cook E H, Jr. (2004) Molecular genetics of autism spectrum disorder. Mol Psychiatry 9:819-832

Wang K, Zhang H, Ma D, Bucan M, Glessner J T, Abrahams B S, Salyakina D, et al. (2009) Common genetic variants on 5p14.1 associate with autism spectrum disorders. Nature 459:528-533

Wang L, Jia M, Yue W, Tang F, Qu M, Ruan Y, Lu T, Zhang H, Yan H, Liu J, Guo Y, Zhang J, Yang X, Zhang D (2008) Association of the ENGRAILED 2 (EN2) gene with autism in Chinese Han population. Am J Med Genet B Neuropsychiatr Genet 147B:434-438

Weedon M N, McCarthy M I, Hitman G, Walker M, Groves C J, Zeggini E, Rayner N W, Shields B, Owen K R, Hattersley A T et al.: Combining information from common type 2 diabetes risk polymorphisms improves disease prediction. PLoS Med 2006, 3:e374.Weiss L A, Kosova G, Delahanty R J, Jiang L, Cook E H, Ober C, Sutcliffe J S (2006) Variation in ITGB3 is associated with whole-blood serotonin level and autism susceptibility. Eur J Hum Genet 14:923-931

Weiss, L. A., Arking, D. E., Daly, M. J. & Chakravarti, A. A genome-wide linkage and association scan reveals novel loci for autism. Nature 461, 802-8 (2009).

Zheng S L, Sun J, Wiklund F, Smith S, Stattin P, Li G, Adami H O, Hsu F C, Zhu Y, Balter K et al.: Cumulative association of five genetic variants with prostate cancer. N Engl J Med 2008, 358:910-919.

1. A method of detecting the presence of or predisposition to autism in a subject, the method comprising detecting the combined presence of an alteration in the gene loci of at least PITX1, ATP2B2, EN2, JARID2, MARK1, ITGB3, CNTNAP2, and HOXA1 in a sample from said subject.

2. The method of claim 1, wherein the alteration is a single nucleotide polymorphism

3. The method of claim 1, comprising detecting the presence of a single nucleotide polymorphism (SNP) at position rs6872664 of PITX1 (nucleotide 301 on SEQ ID NO:1) or any of rs1700488 (nucleotide 301 on SEQ ID NO:9), rs6596189 (nucleotide 201 on SEQ ID NO:10), rs11959298 (nucleotide 301 on SEQ ID NO:11), rs6596188 (nucleotide 301 on SEQ ID NO:12), ss13907917/rs1131611 (nucleotide 201 on SEQ ID NO:13), rs6871427 (nucleotide 201 on SEQ ID NO:14), rs10079987 (nucleotide 201 on SEQ ID NO:15), or ss330962/rs254549 (nucleotide 101 on SEQ ID NO:16).

4. The method of claim 1, comprising detecting the presence of a single nucleotide polymorphism (SNP) at position rs2278556 of ATP2B2 (nucleotide 201 on SEQ ID NO:2) or at position rs17223473 (nucleotide 452 on SEQ ID NO: 17).

5. The method of claim 1, comprising detecting the presence of a single nucleotide polymorphism (SNP) at position rs1861972 of EN2 (nucleotide 301 on SEQ ID NO:3).

6. The method of claim 1, comprising detecting the presence of a single nucleotide polymorphism (SNP) at position rs7766973of JARID2 (nucleotide 251 on SEQ ID NO:4).

7. The method of claim 1, comprising detecting the presence of a single nucleotide polymorphism (SNP) at position rs12410279 of MARK1 (nucleotide 201 on SEQ ID NO:5), or position ss44063993/rs3806329 (nucleotide 301 on SEQ ID NO:23).

8. The method of claim 1, comprising detecting the presence of a single nucleotide polymorphism (SNP) at position rs5918 of ITGB3 (nucleotide 401 on SEQ ID NO:6) or any of rs7214096 (nucleotide 343 on SEQ ID NO:24) or rs8069732 (nucleotide 251 on SEQ ID NO:25).

9. The method of claim 1, comprising detecting the presence of a single nucleotide polymorphism (SNP) at position rs7794745 of CNTNAP2 (nucleotide 301 on SEQ ID NO:7).

10. The method of claim 1, comprising detecting the presence of a single nucleotide polymorphism (SNP) at position r10951154 of HOXA1 (nucleotide 521 on SEQ ID NO:8).

11. The method of claim 1, comprising detecting the simultaneous presence of a SNP at position rs6872664 of PITX1 (nucleotide 301 on SEQ ID NO:1), position rs2278556 of ATP2B2 (nucleotide 201 on SEQ ID NO:2), position rs1861972 of EN2 (nucleotide 301 on SEQ ID NO:3), position rs7766973 of JARID2 (nucleotide 251 on SEQ ID NO:4), position rs12410279 of MARK1 (nucleotide 201 on SEQ ID NO:5), position rs5918 of ITGB3 (nucleotide 401 on SEQ ID NO:6), position rs7794745 of CNTNAP2 (nucleotide 301 on SEQ ID NO:7), and position rs10951154 of HOXA1 (nucleotide 521 on SEQ ID NO:8), wherein detection of the simultaneous presence of C at position rs6872664 of PITX1 (nucleotide 301 on SEQ ID NO:1), A at position rs2278556 of ATP2B2 (nucleotide 201 on SEQ ID NO:2), A at position rs1861972 of EN2 (nucleotide 301 on SEQ ID NO:3), C at position rs7766973 of JARID2 (nucleotide 251 on SEQ ID NO:4), A at position rs12410279 of MARK1 (nucleotide 201 on SEQ ID NO:5), Tat position rs5918 of ITGB3 (nucleotide 401 on SEQ ID NO:6), T at position rs7794745 of CNTNAP2 (nucleotide 301 on SEQ ID NO:7), and T at position rs10951154 of HOXA1 (nucleotide 521 on SEQ ID NO:8), is indicative of the presence of or predisposition to autism.

12. The method of claim 1, wherein the subject is affected with autism spectrum disorder (ASD).

13. The method of claim 1, wherein the subject is a sibling of an individual with an autism spectrum disorder (ASD).

14. The method of claim 1, wherein the presence of an alteration in the gene locus is detected by sequencing, selective hybridisation and/or selective amplification.

15. The method of claim 1, wherein the presence of an alteration in the gene locus is determined by DNA chip analysis.

16. The method of claim 2, comprising detecting the presence of a single nucleotide polymorphism (SNP) at position rs6872664 of PITX1 (nucleotide 301 on SEQ ID NO:1) or any of rs1700488 (nucleotide 301 on SEQ ID NO:9), rs6596189 (nucleotide 201 on SEQ ID NO:10), rs11959298 (nucleotide 301 on SEQ ID NO:11), rs6596188 (nucleotide 301 on SEQ ID NO:12), ss13907917/rs1131611 (nucleotide 201 on SEQ ID NO:13), rs6871427 (nucleotide 201 on SEQ ID NO:14), rs10079987 (nucleotide 201 on SEQ ID NO:15), or ss330962/rs254549 (nucleotide 101 on SEQ ID NO:16).

17. The method of claim 2, comprising detecting the presence of a single nucleotide polymorphism (SNP) at position rs2278556 of ATP2B2 (nucleotide 201 on SEQ ID NO:2).or at position rs17223473 (nucleotide 452 on SEQ ID NO: 17).

18. The method of claim 3, comprising detecting the presence of a single nucleotide polymorphism (SNP) at position rs2278556 of ATP2B2 (nucleotide 201 on SEQ ID NO:2).or at position rs17223473 (nucleotide 452 on SEQ ID NO: 17).

19. The method of claim 16, comprising detecting the presence of a single nucleotide polymorphism (SNP) at position rs2278556 of ATP2B2 (nucleotide 201 on SEQ ID NO:2) or at position rs17223473 (nucleotide 452 on SEQ ID NO: 17).

20. The method of claim 2, comprising detecting the presence of a single nucleotide polymorphism (SNP) at position rs1861972 of EN2 (nucleotide 301 on SEQ ID NO:3).


SEQUENCE LISTING (25 sequences)

SEQ ID NO: 1 - DNA, 601 bp, Homo sapiens
  misc_feature (301)..(301): rs6872664=C/T
ccctccctccctccctctagggttcctgggaactgtccccactggaaagcgcccgccgggtgctggtcgtcagtaggcacctctagtctggtgtgccgcggagaagagcccaagaatcggagctggagccgcgggcctgcgttcctggctgggcagggcctgcacccttagctcggctcagattctgaccctgctgctgatgttcccagcaaatgtatgctttttgtttgtttgttttgcgaactcttagggggtctaaatctgagggggtctctgcttttctgaactaggatcagatctytccagcctaaagtccctccactttcttctcctcaggggtgtatgggagccccactgggcaggcacaacacagccgggttctcggccttgcttccaggcctccagtcccaaagcctagaaaaccccaccaactgcagcccagacagacagactctgtctaggtgacctcgggctttagtctgtctctctttgaagctacacaaaacacacgcagtcatagaaacattaatacataaagaaaaagagtttaaaaacaaaacacagaagcccagaaaagctcacagctctatgcctaaagaca

SEQ ID NO: 2 - DNA, 701 bp, Homo sapiens
  misc_feature (201)..(201): rs2278556=A/G
tggtctctgggctgggggctgagccctcagggagcccttggcccatcccctgcaccctcagggctctgcatcccaggtccagtcctgactccgggtgaaaggagtgaagcaagattagcttttaaaaaaattatgaattatttcaaacatgaaaaagagaatgatgtaacaaagttacgtgcctatcatccagctttgtarcatcttaacattatgccgtacttgcctcagatttttctattttaagaagtaaaacattacagataaagttgaatcccctccgcaaacctctccccatctgcataattcactccttgtcccagaaacaacttctgtcttaaatttagcattaatcattaccatgtgtgtatttaactgttaattccgagataattgtaattcacatgcaataagaaacaatacagagatgccgtatacccttcatccagtttcccccaatgtcatcaccatagtattaacacaagtaccacaactgagatggagatgctgacgcaaggaagccgaacttgtggagagttcaccggctttacatgagcttgtgtgtgggtgtgcgtgtgtattttctatgcaatggcaccacatgtgagttaatgtggccaccaccactgtcaagattcaggacagttcatcacaagggtccttgtactaccctttttttaaagctatggtcacctctctct

SEQ ID NO: 3 - DNA, 601 bp, Homo sapiens
  misc_feature (301)..(301): rs1861972=A/G
agggagtgcttggcattcaccctgggcctccagttcgtcccccacctcttgctgggcacagccccagcaccctagttgactctcctgacctgggcagggtgcagtcccagggcctccaaggagatccacattcctcttctcctcagtgtgcccggcagctctccggccctgaagggtggggggcccccagccttctccagccacagggacctgtgatgaagctggggccagatgctccctaaagccgattcatacaccgcacaaattgaaacccagaggcgaggtcaccactccctgccartggccttgcccccttcttcccccacagggaacgccagggggttgagcctcttatcaccaaaaagaaactgatgacacttccctccttctgctctcctccctctgccctttccccatggatagcaggtcctagaagccttacagcgaccctgcccaaaacctggggcaggtccacagggagaaggccaggtcaggttcataagtctgaatcccagttgggaggcacagtggggagggtcagaagtggacctggacaaggtcagctgggctaccctgctgcccacagtgaagcagtcccatg

SEQ ID NO: 4 - DNA, 501 bp, Homo sapiens
  misc_feature (251)..(251): rs7766973=C/T
ttctagtgaattttctggttgactgtttcaagccatggcattaactccttatgaaggatattttttggctgtcctgtcttcagaagtccggtgtgacgcgcgaactcaatggttgcgttcccgaggaccttatggagtgcttcctgtctgtctgccagtgctttgttactgtgcacctctgtagatatgcagtagggggtttttgggtggtgtgcatgctgatccccagagggtttatattttacctgcaytcctgaggatgtgtttgtgttgcttggggccctgagaaaaaactgcattcagcggaggatttagtaagaagaaagttggttccatgaagtgtacatcagcagcccccgaggatgctgtggggttgcaactcctaaggcttaactgcggcggggatcggttaatagtatggacaaccccacaccccagaacaggggataggtgatggacgacagtgggacaccatacctaatgtgcatgcctgcttcctag

SEQ ID NO: 5 - DNA, 701 bp, Homo sapiens
  misc_feature (201)..(201): rs12410279=A/G
ttctaaacttagcagatataattcagtgcctacagcttcattcgtgggcctcttccaatattctcttttcaagatagcaattccaaatttgtatacttttgagtgactttgcatcttaatgtctcctttaccctccctttagagcaggctgatttggagaaagaatacctggcgagtactgcaaaacaggacagccatcaragattcttccctgatgacatctcagtttcttttttctttttcttttcttctcttttctcttttcttttctctttctttctttctttctttctttctttctttctttctttctttctttctttctttctttcttcctctctttctctctttctttctttcttcctctctttctttctctttctttctttctttctttcttctttctttctttctttctttctttctttctttctttctttctttctttctttctttctttcttctttctttctttttctctctctgtcacctaggctggagtgcagtggtgtgatctgagctcactgcaacctccaactcccgggttcaagcgattctcttgcctcagcctcctgagtagctgggactacaggcatgagctaccacacctggctaatttttgtacttttagtggagatggggtttcatcatgttggccaggccggtctcgaactcctgacctcaagtgatccaccctcctt

SEQ ID NO: 6 - DNA, 801 bp, Homo sapiens
  misc_feature (401)..(401): rs5918=C/T
cacagttaaaaaatgctggtctaccaggcatcttactgtacaggctctccttctagctacaactccatgaataggccttcttgatatctcaatttctcttttctttttcctttttttttttttggtagagacggggtctagctatgttgcctaggctggtcttgaactcttggcctcaagcagtcctcatgccttggcctcccaaagtatcaatttcccctttctgtacaacggtcctaagggattatcccaggaaagaccacaacaatttgtttatgctccaatgtacggggtaaactcttagctattgggaagtggtagggcctgcaggaggtagagagtcgccatagctctgattgctggacttctctttgggctcctgtcttacaggccctgcctcygggctcacctcgctgtgacctgaaggagaatctgctgaaggataactgtgccccagaatccatcgagttcccagtgagtgaggcccgagtactagaggacaggcccctcagcgacaagggctctggagacagctcccaggtcactcaagtcagtccccagaggattgcactccggctccggccaggtagggctgggactctttgcggggagagacctgaagcaggtgggcatagagcacaaggtggaggtctgaggaggaagtcttggggaagtagctcagaatggaaatggggtgggaagacaaggatgaggggggaggtgtgggcaagagaatggaagaaaataagagacgttaggacttgagtcagaagatctgaggtgaatctttactgtatcata

SEQ ID NO: 7 - DNA, 601 bp, Homo sapiens
  misc_feature (301)..(301): rs7794745=A/T
tctttttccttttctttattatatgacatcttatatcaaaaaaaaagaaaaagctaaaaaaaaaacactctgctacatgtaaatctgaactatttattttaggcaaaatggcatgaataaaacaaagctgatctacatttggctttgtcttgtcaaagaagcaatctgaacaaataggctaagatcaattctattagaggtgaaatacggaccaagataccaacattgatcccttcagccatggcccttgcatatagttccaatgtacaataatacaggtcaggacctggaaaggcctaawtgataagactaagtgtcaaaatcagtgttgaaaggctgctcaaactacagaaaatgaggtatccaggcatttaataagcatgcattgagctaagcacataccaccactaaagattcatttgcatgtgggattaaaacatctgaaaagttgacaagggaacgttttagagactctgagatggtgacatcattatgacacaaggcactgttggtctgaagcacactggtgagtgtccccaaccataaatttggagatagttcaggtggcatctgagtattaatcaaaagaaaatagatactt

SEQ ID NO: 8 - DNA, 1041 bp, Homo sapiens
  misc_feature (521)..(521): rs10951154=C/T
taacagaacactaccgtcactggaaattaagcataccagctccttccagaataatcaaggagcatccaccaaccagcaggactgacctgttttgggagggtttcttttgactttcatccagtcaaaagtctgcgctggagaagatgtctccgatgcgggggagcgacaggcttcttggtggctggcgtggagaggggacaaggagttattatacgtagccagggccaggctctggtgctcctgtccatatgagtggtgaatgtattgaggcgagcccaccgcgcccccagcataaccctggtggtggtggtgatgctggaccatgggagatgagagatttccagagtaaacagcgggagcgcactgggggtacccaccacttacgtctgcttcctgatttaacgcgtaggggctgtaaggcgcactgaagttctgtgagccatagcttggaccacaacttgagtgggagtaggacacccccaggttcccggaagtctggtaggtagccggctgggggtggygatggtggtggtggtggtggtggtggggcgaaccgatctgcacccccctgcccactaggaagcggtcgtcgccgccgcaactgttggcgctgaccgcgcacgactggaaagttgtaatcctatggtccgaggggtaggctcgggctgagcaggtccccgagtcgccactgctaagtatggggtattccaggaaggagttcattcttgcattgtccatctgtcactgagtgacctggtcctgcgaagcccggcgtgactgtgccaactttctcacttcctccatggggccggagaagaaaaatgatatgaatgtacagtgcgcaagagggggggcgggagggcggaggacgctggaggaggggcacgtgacggtgtcagccaatggctgagcctcctgcaaaagtttgccggcttccgcagtgatggatcaccgttttagtggcatttaaatccccggcgctccgccgtctaggtgacgcgcagtcgcccccccaggcagcgttgtaatcctatggtccga

SEQ ID NO: 9 - DNA, 601 bp, Homo sapiens
  misc_feature (301)..(301): rs1700488=A/G
ctctcctgggaagaaaaagcccgacctgtgccggcgagagcaggctgccttgggctgggggcacctccgagcgctgcggcgggacgtcggggtccggggcgaagagagccagggcgcggaccgacgtctgctgcttttctgcggcattgctgcccgaacgaacgaacgaacgaacgaacgaagcggtttcgtttaggaaaaataccctcttgacgcgaagccacggctgaagtcccgggccacgcagaggggccagcaattccatgggtggtggggccctccatccctggacgcagcgggragcagcgggcggccctctctcctgcccacttctcctgggggtgggggagccctgtgtcacccgacgctgcgaggggctgccacgggcttctcaggttgtgcccttcgcggggccgtggcccgtgcccccgaggctgctaccctcttgaggtgcccggagccaaagcagagggttgatcccgccggttgggtgaggtagcacccggtgacggcggctatgactgtgccctgggcgcaggaacgagctcctcgcgctgcctcgaaggctcctgaccttccgcgccttgcgctgtcccgcgcc

SEQ ID NO: 10 - DNA, 401 bp, Homo sapiens
  misc_feature (201)..(201): rs6596189=C/T
aagctccgtacaaacacacccctaactaaatacagagctgtaagtgtataaactccaggttgacagaagcatttatttctctgggatttaaaaaaataatgtaccgttgtagatagatttttaactccttcccaataaggcccttgtagagataatttttaaatcccagagaaacacattatagttagataatttaaattycccaaataacaccactgtaaatggattttgagaaaattccggagaaacgcgccattttggattgactttttaaaatctccggaagaataatgttgcatgtagatattttaaattccagacctacacttcgtacgttaattctttcagaatcgaaagatacagttaaaattaattcatcctcatagatttggttcaaaaaa

SEQ ID NO: 11 - DNA, 601 bp, Homo sapiens
  misc_feature (301)..(301): rs11959298=A/G
ccccgttgcaccccgtcccggctccaccgaaccgctcgccaccagcgctggcggtcttccctccctccctccctctagggttcctgggaactgtccccactggaaagcgcccgccgggtgctggtcgtcagtaggcacctctagtctggtgtgccgcggagaagagcccaagaatcggagctggagccgcgggcctgcgttcctggctgggcagggcctgcacccttagctcggctcagattctgaccctgctgctgatgttcccagcaaatgtatgctttttgtttgtttgttttgcgarctcttagggggtctaaatctgagggggtctctgcttttctgaactaggatcagatctctccagcctaaagtccctccactttcttctcctcaggggtgtatgggagccccactgggcaggcacaacacagccgggttctcggccttgcttccaggcctccagtcccaaagcctagaaaaccccaccaactgcagcccagacagacagactctgtctaggtgacctcgggctttagtctgtctctctttgaagctacacaaaacacacgcagtcatagaaacattaatacataaagaaaaa

SEQ ID NO: 12 - DNA, 401 bp, Homo sapiens
  misc_feature (201)..(201): rs6596188=A/T
aagggccacaaattctcaaaatcagacaggaagaaacacagagcaaatgtaaattaagaagccagaaagctccgtacaaacacacccctaactaaatacagagctgtaagtgtataaactccaggttgacagaagcatttatttctctgggatttaaaaaaataatgtaccgttgtagatagatttttaactccttcccawtaaggcccttgtagagataatttttaaatcccagagaaacacattatagttagataatttaaattccccaaataacaccactgtaaatggattttgagaaaattccggagaaacgcgccattttggattgactttttaaaatctccggaagaataatgttgcatgtagatattttaaattccagacctacacttcgtacg

SEQ ID NO: 13 - DNA, 401 bp, Homo sapiens
  misc_feature (201)..(201): ss13907917 =G/T
gctcatggagttgaagaaggtgaagctcttggtggagagcggcgctggcgccaggctcttggcggcccagttgttgtaggagtagccggcggcgtacacgtcctcgtagggctgcactaggccgctgaactgcggcacgtagccacccttgcacaggtccagctgctggttacgctcgcgcttacgccacttggctcgcckgttcttgaaccagacctgggggaggggacgggagaagggtcagggccgctgcgggccgggagggaccccacccccttccccaccgcctggagccttccgtcggcccgctgccctccgaacgtcgtttctctccttcgaccgatatttccgctcctgcattccctccacaatggttcctttcctgtccccagaaaatacta

SEQ ID NO: 14 - DNA, 401 bp, Homo sapiens
  misc_feature (201)..(201): rs6871427=C/G
tggacgctgtctggttcctgagaatggctgagctgtggggaagaagactaggaaggaggtagcagctggtcgcacaactggggagcagaggggcaacttatggatgcaccatagcctcctcctcccagcggctgctttcatgggagaagagcaccctgagcctgtctggaggcctctgcatcctagagtccagacagcctsgtctccaagccccagcccacctcttttcctttccctggaagagatgcgggaggccgaaagcagccccgggaaagggcacggggacagcaagtctcctgtgctgcccctgctgcctgtagcttttgccctgactaccgtagacctgaaccctagacctgaatcctcccccagtggacagcaacctgggatttcctccctct

SEQ ID NO: 15 - DNA, 401 bp, Homo sapiens
  misc_feature (201)..(201): rs10079987 =C/T
aggccaaggtgggaagcacagtgcgtctctgggtagagacttagaaatgaatggtctgtctgtgggcctctagaggaggttaagactcacagccttctcacttgccttctcacttcccagagggaccaaagtgactctttgggccatcccccaatggggactattctgtgagcagcactcactgcttagtctgaggggcgygtttcctcactgttagtgcatttaatgcagttattgagcacctgctgtgtgcccctctgggcccaaacctctggcagggctccaggcaggcacaatagtccctttatttggtgatcttgatgcctgctgtgtagaaggaaaagccctcagaccatttcaggtgaagccccatatcctcaggggacctgagacccaaagag

SEQ ID NO: 16 - DNA, 201 bp, Homo sapiens
  misc_feature (101)..(101): ss330962=A/G
gtttatctgccctcatcccctgcttaggtgctaggaagtttggtccaggaaagctgggcaggctggtgcgtggggcacaaccagggcctggaagtcaggargttcttagtgaaacttggggggtacaaaacacattttttataatcttattttctctcctctaaaaatatatgtatatattttttgcaatttccctttagt

SEQ ID NO: 17 - DNA, 652 bp, Homo sapiens
  misc_feature (452)..(452): rs17223473=C/T
ttgcccaggccactcagcaagcggagccccactttgatgtattatatactcacaagattcatctgaaataacactcccacagctgaaatggtttacaaagcacataaaactcaggctagtagttcttgaccctgcctgcacattagaattgcttggaggagcctttaataaatacccacgtgcgagccttgccccagaggttccagttcaatttgtctggggtgaatccaggaatcaatgtttttttaaacactcctcagagtgattccaatgtgtacccagggctgagaaccactgggggctagactaattgcctcaatgtacaagtggggaaactgaggccagagaggggaagggatttgcttagttcaccaggcttatttccagcactcctctggagtcagaggagtgtggctaataatgaaaatagctaccatcaaccaactctacgytaaaggaaatgaaataagagagatctaagtgtgacaatgagtagaacttcctgatgaaccagcggcttagggaggagaagggggaggactctggctatagaaatgtttaagaggatatagtggaaaacacagtggccaaggggcatgcaagtgaagatgagtcttgctgggtgaccttgggcagggccctctgtggctct

SEQ ID NO: 18 - DNA, 601 bp, Homo sapiens
  misc_feature (301)..(301): rs12521388=A/G
ggctgtcaattaacctaaaatttgtcctcccagtgcccatcccgtttatagttatttaggctttgtaatgactgggggatagaaagatgttcagtcatttttatttctacctcccagattggacctgttacaaactcagcctcaataagccttgtcgttgactttagggactcagtttctccccagggtggatgggggaaacggtgccttcaagaggcttcaccaaatgtactagaaggccattggccattctattctggcaaggctgagtagaagatcctaccccaattctttacaggartataggcctgtctaaagtgagctccatgggcagagctaccccttattattccggacctgcagtcacttcgtgggatctgcccctccctgcttcagtacccaaatcctttccagctataacagtagggatgagtacccaaaagctcagccagccccatcaggattcttgtgaaaagagaggatatgttcacacccagcttcagtattttccctgccaggagttttaggtctcttcctctctcagagctacttgggctatagctcctgctccacagccatcccggccttggcatctagagct

SEQ ID NO: 19 - DNA, 501 bp, Homo sapiens
  misc_feature (251)..(251): rs12518194=A/G
tgtaaacatgaattctatgagacactttgtccagtttcaatatttatattaatacagactgaatttattctaaagaagatctatgtatttaccaagccgtttacacaggatgcttactaacttgttgggatctgtggaccaatttttcagagatatattaaatatgttaatggcctccaatcttgttaaagcccaatttatttttccttccattatcttctatgtggcatataaacagaggatctggggcrtacaacttgatttcaactttttacactggttaatgcccttggcttattgtatctggtgtaccaaccaagcacacactatggcagcatcaccataaatgatacatgctaaaaaattgtttttcaaatatcagaaagggaattagttaccttttagtaattagagacacaacattgactacccttatgttatcctcaaggtatctgttgttgtaagttgacagaaacctagcctatcaaagttgctaaatgg

SEQ ID NO: 20 - DNA, 601 bp, Homo sapiens
  misc_feature (301)..(301): rs10942147=A/G
agattgaacgggagaaggagctgatggggaggctgaagcagatcgaggaacagactaagaaggctcagcaagaactggaagaacagacccgtagggctctggaacttgggaaggaacggaagcatgcccagagcgaggctgaaaagctggccaaggagcttcaagaagctgaagaggccaaggaagtcttgctgcaggcctcccgggaccagaaaaagactcaggaatagctggccttggaaatggcagagctgacagctcaaatctctcagctggaactggcccgacagaagaaagagartgaggctatggagtggcagcagaaggcccagatggtacaggaagacttggagaagacccgtgctgagctgaagactgccatgagtacatctcatgtggcagagcctgctgagaatgagcaggatgagcaggatgagaatggggcagaggctagtgctgacctacgggctgatgctatggccaaggaccgcagtgaggaggaatgtaccactgaggcagagaagaatgagcgtgtgcagaagcacctgaaggccctcacttcggagctggccagtgcccgagatgagtccaagaagactgc

SEQ ID NO: 21 - DNA, 601 bp, Homo sapiens
  misc_feature (301)..(301): rs7731520=A/G
ggcttgagggaagtttgtttctttggtctgcagtaccaggacaccaaaggtttctccacctggctgaaactcaattaagaaggtgactgcccaggaagtgcggaaggaaagccccctgctctttaagttcagtgccaagttctaccctgaggatgtgtccgaccgaggaattgattcaggacaccactcagcgtctgttctttctgcaagtgaaagagggcattctcagtgatgatatttactgccggcctgagactgctgtgctgctggcctcccatgctgtccagtctaagtatgggarcttcagtaaggaggtgcataagtctggctacctggccggagacgagttgctcccacagagagtcctggaacagcacaaactcagcaaggaccagtgggaggagcggatccaggtgtggcatgaggaacaccgtggcatgctcagggaggatgctgtcctggagtatctgaagattgctcaagatctggagatgtatggtgtaaactacttcagcatcaagaaaaagaaaggctcagagctgtggctgggggtggatgcactgggtctcaacatctatgagcagcatgacagactaactcc

SEQ ID NO: 22 - DNA, 5553 bp, Homo sapiens
  misc_feature (5047)..(5047): rs4327572=C/T
tagtaaatttgtttacattcctctataatatggtgtgcaatcataaagttgaacacacacttcctttttctgattaaattcaagtaccttttcatcaggattaggttatctaaatgagtttcctctaaggagaatcaatcatactgcaattaggttttggtttgatttttaaaaaaccttttttggcaactggcctgggaaatacagattttgtaataccctgtgcttcttgctgtctttattcaggtctttattttattacttaagaaagctgagctttgaaaaagttaagattgttgttgttgttgttcatccatgtaactttctgtatttacttcttattctggttaagtgaatgactattattttacaataacttatttgatcaagtgtttctaaatgtttggtatctttgcttggttgcccagtatcaaaattttaagaccttttttttaacctcaagttaactttgggatatgttaaaaggccccagtacttctcaaatgtttacaaaaaagagatattgaactatattggatttatctgaaatattgagttacatgtaaagacttgtcaaatgataaaagatgctagatcttctttgaattacatttatgtatacattattgatattaatgttccaaaaatgtataaaatatttaaaaaatctaatatgtcatgataatttgggttatgtggcgtcacacaaaattaaccacatttccttgtcagatttcagtttttgttatttatagttattgttttgaattatctaaatgattctgccattaaattcatggaaacctctataatagtactcttgaatgcagatttctaaaaactttacaaccaatagactaaagaaaaatgttctagaatattaatgaagaaattgatgaacttgtgaaactgcaaatccatatcaaccaaaacaaaaattaactacatcagtgttgtgggaagtcagggacccctaacagagggaccagctgatgccatggcagaagaacataaattgtgaagatttcatggacatttactagttccccaaattaatacttttataatttcttacgcctgtctttactgcaatctctgaacataaattgtgaagatttcatggacatttatcacttccccaatcaatactcttgtgatttcctatgcctgtctttaatctcttaattctgtcatcttcgtaagctgaggatatatgtcgcctcaggaccctatgatggttgtgttaactgcacaaattgtttgtagagcatgtgtgtttgaacaatatgaaatctgggcaccttaagaacaggataacagcaattttcagggaacaagggagataaccttaaagtctggctgccttaaagtctgtcctgcctgtgggcagggcaggacagagccatatttctcttattaccaaaaatgggtaagagaaatatcgctgaattctttccccagcaaggaatattaataattaacagccctgggaaaagaatgcattcccagggtggggcctctaaaatggccaccctgggagtgtctgcattatgcagatgtagatagggatgaaacatgccctagtatcttgcagcactcccaggtttgctaggattaggaaattccagactggcgaattctagtcagactggttctctgttcttgaaccctgacaatgtgtgcacaacaggacagggaagttcattagtgattctagttttgccctgaccttctgccttgtgatcttttgtcacccttgaagcatgtcatccctttgaaaattgctaataaaaacttgctggttttacagctcagggggcatcatggaacctgccaacatgtgaggtctagcccggacacccagctttaaaatttctctcttttgtactctttctctttatttctcggaccggccgacacttagggaaatagaaaagaacctatgtgaaataacgttgaattatcgggggcggattcccctgatgcatcagattaaatcactgctaaagataatgtttttgtgacttttatttgaaacatatctgttctttaatcttttgttttccagatttaaagaaactttctctttcaaattatgtggagtttacagtatatctttatgaacaaaagtgggagcatttacttgttctccctacttgatccttgcaaaatgtagaaactattcatgagtattcttattttctatggcaatattgttatttgcagaagttcaatcacaatatgctctctttataacaggatacaaatggaaacattggttatatcaccaagactttgattagaatgttatatttgggaatatgcatagactgcctagcttcaagggttctgagcctcacagtgagtgaatagaaaattgtgacctcttggaagtcccaggaacctcaagatattaggtactgcagatgaaattctaaaatctttcttgatttggctttttagtcttaaaaaaagtttaaatctgagattacaatgtgttcaaatcactattctttcaggccaaatttgataaccctaaaattttacaaacaaatttgtttgattgtcttaggtaaataatgggcaagattatagagagaaaaagttgtctttctaaagaaaaaactatatctattattagattctagctctgttcattgtttttgaggtcttattatctacctatagaatggactacaccctgaagtcttctagatttcttcaatccaactatgtttcatgtaatgactaagaacagggactgttctggttctcaaagccctgtaagctgaaactaaatatattttaaggaacaagtctcatgtcttatgcatgggtcacacaactatttcaccaaactacctgataccataaccagagatatttaaactatacaccaggatgagaagttgatagcttcatgctgtgcacagctctttctgtgacattggagcaggactccatattataatgagactcttactttccttaaggctacctttttcaattgtctggataatggtagagttgaaatttcacaattagtatcttctgctggtatcctgacagaacctgacctaaaagatcctttagactaccacctagaaggtaactttgaaaacacccataatacaactgtcacttactatctgctctaattcaacccagtcatgggatactagattaaaaatttgctcaatattatgatgtagtttagatttttgttttctccaaatctcatgttaaaatgttatccccagtgtgggaggtggggcctggtagaggtgttttggtcatggaggtggatcctttataaatggcttgctgctgtcttcctgattgagagggaattctcacaagatctggttatttaaaagtttgtggaacctgccccctctctctcatgctcctggtttcacaatgtgatgtgcctgctctcacttcaccttctaccttgagtaaaagttcactgagtcctcctcagaaacaaagcagatgtcagaaccatgcttgttcagtctgcaaaaccgtgagccaattaaacccgctttctttataaattacctagccttaggtatatctttacagaaaaacaaaaatggcctgaaagttatctattaactaactaactaaataaatatctggccagactcggtggctcatgtctgtaattccagcacttttggaggccaatgctggaggatccctctagcctaggagtttgaaaccagccctgtctctattattattatttttttaaattagctgggcatagtggcatgtctgtggtctcagcaactcaggaggctgaactgggagaactgcttgatcccaggaggttgaggctgcagtgagagctgtgattacaccactgtattttcagccttggagtacagacttgtctcaaaaaaaaaaaaagtctgtgctattgctaatatttcatgctgtacctaaataaattcctcatggaaagttgagacccatatacacaaaataagaaagtagggcacatggttaaaatagatgccacctaattgcttattctcatttgatttattgaatttgttgcctttaagcgtaggttcatagctcaaacccattaggcaaagtggaactatgttctcattatcaatttcacttcatattttttctttttaatttttaatgttgtaactgttatttgttaaatgtatgtaaaactacaactcctaactgaataatgctggcccagcattttaaggtaatagacaaacaaaattgaagttaacactgaactttaggtagacttagcctgagagccactatttctgaatctcccttgtaactcaaatgtggcaaaaaaggttttgacacaggctgctagttgccaactgcaacctccagtgtggggctagagcaacaacaacctgggacaggttcatcccagtaccaagggacatcaaacataactacagaatgactgatcagtggtgctttcagagaaagaccctgaccaaaactgtgaaatatgaatgttgtcagaataaaaatggaatcattagtgctaaaaaatgctgataaatgaagccagataaggccatgaagggaaggttcccatgcacaaatatctggtaacaagaactatcacaagagacgacaacaaattcaacttttcacaaaagccaccataaacttaactaaaaatatacttacgtgaggacattttcctagcaactttctgtccaatctcagactgttgccaatttgttattgatccttgcagccaaagataattatctcaagaaaataattatcctcatttttcctttaaaaacctttgttgtcctttgcctccctgtatatgcacagtttattatggcatgtatattcccattgcaatgtcaattcccaaataaatatctccctgtttgttttataggtcgacacatgcatacatatttgattcaccatattgacatattaaatggaaaaaacccaattacatgagaagttgctgaataagtaatttgataaaattatttattttataaatacttataaagcaaayaaaacagcaaaatatgaaaaagacaggataagcatgctactatatgctactatatactaaaataaaaaagcatttataaaaaatgctgaaaatggccaggcttggtggctcatgcctgtaatcctagaactttgggatgccatggtgggtggatcccctgaggtcaggagttcaagatcagcctggccaacatggtgaaaccacgtctctactcaaaatacaaaaaaattagccggatgtggtgccacacgcctataatctcagctactcgggagactgagacaggagaatcacttgaaaccaggaggcggaggctgcagtgagtcaagatggcgccattgcactccagcctgagcaagacagagcgagacaccctctcaaaacaaaataaaaaaaaaatggtgaaaatgtcaaagatatcactattactaaatcaattcattttattacctactttagtcagtgaaacaggaagccgaaaattccccaaacgggtcttccttttc

SEQ ID NO: 23 - DNA, 601 bp, Homo sapiens
  misc_feature (301)..(301): ss44063993=A/G
actgcgccatatgctggcatgtctgcaccagaatttcctttgcgcctccctgttggtttgctccactttttcactgagtcagaccgttgaacaccgtggacacactgtcttgcgtttccgaatatttcctagaatacggacgtttcctaagactcacgataaagattttctgatcgtctctccaaaaccttgccaccaatttgcactcccacgaatcctgttaccgtgactatctcgccatgccctccctagcactgagcgtgatctctagtatcattttccatcgttgctaatttgaacrtgagcagatggagtcctattatttggggtcattaatttcgtagcaagtgcagttgaaggtgttttgcatgttcattgtgcagtgcgcgccgtagtctgcacagtttggccggcaggtgggatgaagggcggggctggcggagcgcgcccgccgcctggtaggccagttcggagcggagccaacgctatcccgggccccacggccagggggcgctgcggcccccccaatcccccgccccgtccgggctggggcggaggagcgggcggggaccaaaggttggtgtctttgcgctcggacctt

SEQ ID NO: 24 - DNA, 571 bp, Homo sapiens
  misc_feature (343)..(343): rs7214096=A/G
gtaaggttagtaataattttacatgagtttatctttgtatcacaagagaccgggaaaagagtagtatatatgcaagacattggagtgtgcttgaacaatgtctggaaaatggtgtaatgctcttgttaatgttagcactattttgttaatcactgttactactattggtgctactgttattttttggattctagatctttgctatccaatatggtaggctttagccacatgtggctatttaaattaaccaaaattgaaaaattaaaaattcagttcttcagctcatcagtggccacatgtagctagtggctgctatattggacagatctagaacatttcaacrtgatagaaagttctcttggatagcactgccacagagagtccctgctgacagattggggattataattttaagtccttctactcagcagggtactgagttgtccttctcattgcaaaattcagtaaaatcttgaggcccactgataatctcaaactagtttctccttgccccaagaaccactcctggggtactttcccagctagaagttgaactaatgttaatgtagtc

SEQ ID NO: 25 - DNA, 501 bp, Homo sapiens
  misc_feature (251)..(251): rs8069732=C/T
cctagggaatgagtgtgtctgagggaatggctatgtttgtggctgggatggaaatttggagctggcaagagctgatggggtgggggtggaagatgaatgtggttgaagagattcggtgattatagagtctcaggctgagtcactggcctggccagcaggtggttacctgagttcctggcctggctgcggctcagctacctgaggcttcctctgtcccacaacaagtcccagccctctccagttttatgacygctccaatccagccctcagtctctctgtctgcttctttttcagatctgctgagtttcctaaaaaggcaacctccctggccgcctggggggaggggcgggagctgggaggctgtgtttatcaccggccaggagggctgccagagcagcagaggccagctccgcacgttgctttggcctctttgccgttcagcacgggaactgcttccagaggcagctgcccaacatctgggccacattaacttcatttagc